(19) United States
(12) Patent Application Publication
(10) Pub. No.: US 2023/0314340 A1
(43) Pub. Date: Oct. 5, 2023

(54) NOISE REDUCTION FOR SENSOR APPARATUS

(71) Applicant: Medtronic, Inc., Minneapolis, MN (US)

(72) Inventors: Luling Wang, Mason, OH (US); Anna M. Belle, Scottsdale, AZ (US); Mohsen Askarinya, Chandler, AZ (US); David A. Ruben, Mesa, AZ (US); Omid Mahdavi, Tucson, AZ (US); David A. Anderson, Stanchfield, MN (US); Shawn C. Kelley, Shoreview, MN (US)

(21) Appl. No.: 17/707,241

(22) Filed: Mar. 29, 2022

(51) Int. Cl. G01N 21/93; A61B 5/1495; A61B 5/145

(52) U.S. Cl. CPC G01N 21/93; A61B 5/14865; A61B 5/14532; A61B 5/1495

(57) ABSTRACT

An analyte sensor apparatus for detecting an analyte in a target environment includes a plurality of biotransducers and a controller. The plurality of biotransducers are configured to provide a baseline signal, one or more analyte signals, and at least one error condition signal. The plurality of biotransducers at least one reference biotransducer, one or more working biotransducers, and at least one working as reference biotransducer. The controller is operatively coupled to the plurality of biotransducers and is configured to receive the baseline signal, the one or more analyte signals, and the error correction signal. The controller is further configured to determine and/or output one or more adjusted analyte levels using the baseline signal, the one or more analyte signals, and the error correction signal.

### FIELD

The present disclosure relates to, among other things, analyte sensors.

### TECHNICAL BACKGROUND

Analyte sensors may be used in a wide range of applications such as, for example, point-of-care monitoring, environmental monitoring, food control, drug discovery, forensics, biomedical research, etc. Analyte sensors may be used to detect a substance of interest (e.g., an analyte) in a target environment. Analyte sensors generally include biotransducers to provide signals that can be used to determine the presence of an analyte in the target environment. Such biotransducers may be subject to noise in the target environment, sensor drift, reference variation, or other effects that can impact sensor response and accuracy.

To ensure accurate sensing of an analyte in a target environment, analyte sensors may be periodically calibrated or replaced. However, in some applications, calibration or replacement of the analyte sensors may be burdensome and expensive. Furthermore, while calibration may correct some sources of error such as sensor drift, calibration may not correct for noise in the target environment.

### BRIEF SUMMARY

As described herein, accurate and reliable analyte sensing can be achieved using analyte sensors that include one or more working as reference biotransducers. Generally, analyte sensors include at least one reference biotransducer to provide a reference signal and one working biotransducer to provide an analyte signal used together to determine the presence of an analyte in a target area. However, the reference biotransducer may be subject to variation, noise, drift, and other effects that can negatively impact the accuracy of analyte sensors. To correct for such effects, a working as reference biotransducer can be used to filter noise and to correct for drift and variation of the reference biotransducer. Accordingly, apparatus, systems, and methods that use working as reference biotransducers may provide more accurate and reliable analyte sensing.

Described herein, among other things, is analyte sensor apparatus for detecting an analyte in a target environment comprising a plurality of biotransducers and a controller. The plurality of biotransducers are configured to provide a baseline signal, one or more analyte signals, and at least one error condition signal. The plurality of biotransducers comprises at least one reference biotransducer to provide a baseline signal of the target environment, one or more working biotransducers to provide an analyte signal based on a presence of the analyte in the target environment, and at least one working as reference biotransducer to provide at least one error correction signal of the target environment. The controller is operatively coupled to the plurality of biotransducers. The controller is configured to receive the baseline signal, the one or more analyte signals, and the error correction signal. The controller is further configured to provide one or more analyte levels based on the baseline signal and the one or more analyte signals. Still further, the controller is configured to provide an error correction factor based on the baseline signal and the at least one error correction signal and output one or more adjusted analyte levels based on the one or more analyte levels and the at least one error correction factor.

In general, in one aspect, the present disclosure describes an analyte sensing system comprising an analyte sensor apparatus for detecting an analyte in a target environment and a computing apparatus. The analyte sensor apparatus comprises a plurality of biotransducers configured to provide a baseline signal, one or more analyte signals, and at least one error condition signal. The plurality of biotransducers comprises at least one reference biotransducer to provide a baseline signal of the target environment, one or more working biotransducers to provide an analyte signal based on a presence of the analyte in the target environment, and at least one working as reference biotransducer to provide at least one error correction signal of the target environment. The computing apparatus comprises one or more processors and is operatively coupled to the analyte sensor apparatus. The computing apparatus is configured to receive the baseline signal, the one or more analyte signals, and the at least one error correction signal. The computing apparatus is further configured to determine one or more analyte levels based on the baseline signal and the one or more analyte signals. Still further, the computing apparatus is configured to determine an error correction factor based on the baseline signal and the at least one error correction signal and determine one or more adjusted analyte levels based on the one or more analyte levels and the error correction factor.

In general, in another aspect, the present disclosure describes a method for detecting an analyte in a target environment. The method comprises receiving a baseline signal of the target environment, one or more analyte signals of the target environment, and at least one error correction signals from the target environment. The method further comprises determining one or more analyte levels based on the baseline signal and the one or more analyte signals. Still further, the method comprises determining an error correction factor based on the baseline signal and the at least one error correction signals and determining one or more adjusted analyte levels based on the one or more analyte levels and the error correction factor.

Advantages and additional features of the subject matter of the present disclosure will be set forth in the detailed description which follows, and in part will be readily apparent to those skilled in the art from that description or recognized by practicing the subject matter of the present disclosure as described herein, including the detailed description which follows, the claims, as well as the appended drawings.

It is to be understood that both the foregoing general description and the following detailed description present embodiments of the subject matter of the present disclosure, and are intended to provide an overview or framework for understanding the nature and character of the subject matter of the present disclosure as it is claimed. The accompanying drawings are included to provide a further understanding of the subject matter of the present disclosure and are incorporated into and constitute a part of this specification. The drawings illustrate various embodiments of the subject matter of the present disclosure and together with the description serve to explain the principles and operations of the subject matter of the present disclosure. Additionally, the drawings and descriptions are meant to be merely illustrative and are not intended to limit the scope of the claims in any manner.

### BRIEF DESCRIPTION OF THE DRAWINGS

The following detailed description of specific embodiments of the present disclosure can be best understood when read in conjunction with the following drawings, in which:

FIG. 1 is a schematic diagram of an embodiment of an analyte sensing system;

FIG. 2 is a schematic block diagram of the analyte sensing system of FIG. 1;

FIG. 3 is a schematic block diagram another embodiment of an analyte sensing system;

FIG. 4 is a flow diagram of an embodiment of a process for detecting an analyte in a target environment; and

FIG. 5 is a flow diagram of another embodiment of a process for detecting an analyte in a target environment.

The schematic drawings are not necessarily to scale.

### DETAILED DESCRIPTION

Reference will now be made in greater detail to various embodiments of the subject matter of the present disclosure, some embodiments of which are illustrated in the accompanying drawings. Like numbers used in the figures refer to like components and steps. However, it will be understood that the use of a number to refer to a component in a given figure is not intended to limit the component in another figure labeled with the same number. In addition, the use of different numbers to refer to components in different figures is not intended to indicate that the different numbered components cannot be the same or similar to other numbered components.

Accurate and reliable analyte sensing can be achieved using analyte sensors and/or systems that include one or more working as reference biotransducers as described herein. Using a working as reference biotransducer, noise that may distort a baseline signal can be filtered out and reference biotransducer drift and variation can be corrected. Accordingly, apparatus, systems, and methods that use working as reference biotransducers as described herein may provide more accurate and reliable analyte sensing.

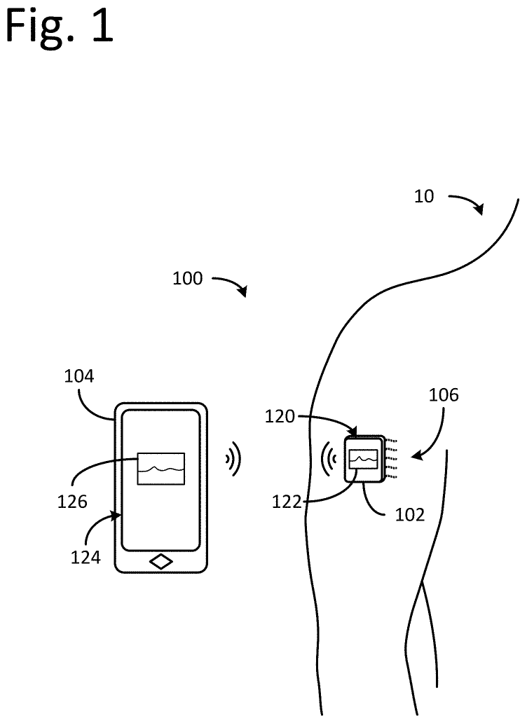

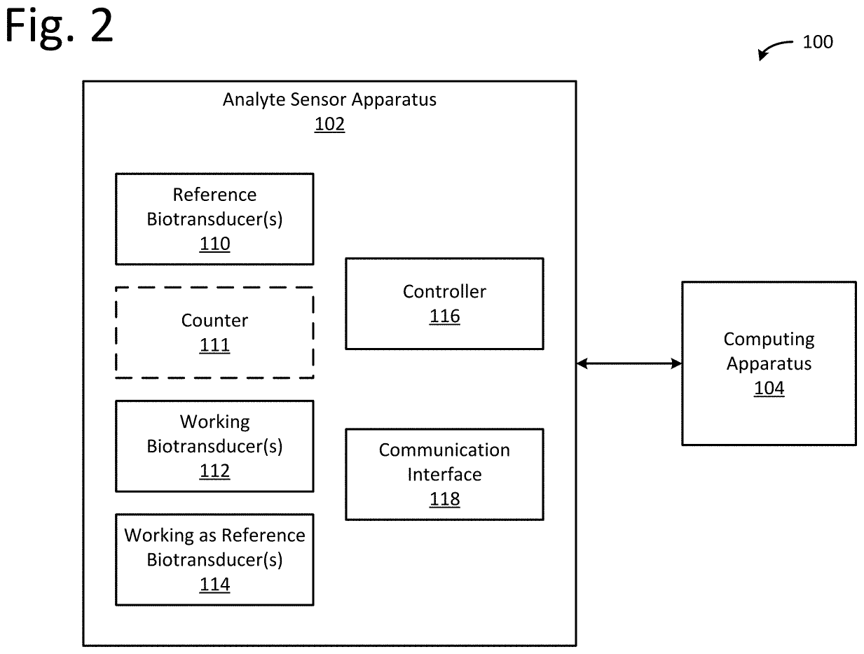

An analyte sensing system is depicted in FIGS. 1 and 2. FIG. 1 shows schematic diagram of an analyte sensing system 100 including an analyte sensor apparatus 102 and a computing apparatus 104 during use. FIG. 2 shows a schematic block diagram of the analyte sensing system 100.

The analyte sensing system 100 includes the analyte sensor apparatus 102 for detecting analyte in a target environment 10. As shown, the target environment 10 is a person or patient. However, the target environment 10 may include any environment where detection of analyte levels or concentrations may be desired. For example, the analyte sensor apparatus 102 may be used to detect analytes that may include reagents in chemical processes, pollutants (e.g., atmospheric or aquatic), health markers (e.g., cholesterol, triglycerides, iron, vitamins, etc.), any substance of a comprehensive metabolic panel (e.g., substances in blood including glucose, calcium, sodium, potassium, carbon dioxide, chloride, albumin, total protein, alkaline phosphatase, alanine transaminase, aspartate aminotransferase, bilirubin, blood urea nitrogen, and creatinine), etc. Accordingly, the target environment 10 may include, for example, a manufacturing facility, a body of water, a particular geographical area, a patient, human samples (e.g., blood, urine, saliva, etc.), environmental samples (e.g., air, water, soil, vegetation), cell cultures, food samples, etc. Furthermore, the analyte sensor apparatus 102 may be an implantable medical device, a remote sensor, a wearable device, hospital equipment, lab equipment, etc.

The analyte sensor apparatus 102 includes a plurality of biotransducers 106. Each of the plurality of biotransducers 106 may be configured to provide signals based on the target environment 10 that can be used to determine an analyte level or analyte concentration in the target environment 10. Each of the plurality of biotransducers 106 may convert one form of energy (e.g., chemical, optical, mechanical, etc.) to an electrical signal. Signals provided by the plurality of biotransducers 106 may include baseline signals, analyte signals, error condition signals, or other signals that can be used to determine an analyte level in the target environment.

The plurality of biotransducers 106 may include any suitable type of biotransducer. For example, the plurality of biotransducers 106 may include one or more electrochemical biotransducers, optical biotransducers, electronic biotransducers, gravimetric/piezoelectric biotransducers, or pyroelectric biotransducers. Furthermore, each of the plurality of biotransducers 106 may include one or more bioreceptors (e.g., nucleic acids, cells, antibodies, enzymes, etc.), electrical interfaces (e.g., field effect transistors, nanowire arrays, nanoparticles, electrodes, etc.), optical interfaces, etc.

Electrochemical biotransducers may include a biorecognition element that selectively reacts with a target analyte and produces an electrical signal that is proportional to the analyte concentration. Electrochemical biotransducers may be used in amperometric or potentiometric sensor apparatus.

Amperometric sensor apparatus may be used to detect a change in current as a result of electrochemical oxidation or reduction. Typically, a bioreceptor molecule is immobilized on one or more working electrodes (e.g., gold, carbon, platinum, etc.). A potential applied between the one or more working electrodes and a reference electrode (e.g., silver/silver-chloride) may be fixed at a given value while the current is measured with respect to time. The applied potential may be the driving force for an electron transfer reaction. The current produced as a result of the applied potential may be a direct measure of the rate of electron transfer. Accordingly, the measured current may reflect the reaction occurring between the bioreceptor molecule and the analyte where the current is limited by the mass transport rate of the analyte to the electrode.

Potentiometric sensor apparatus may be used to measure a potential or charge accumulation. Biotransducers used for potentiometric sensing may include one or more working electrodes (e.g., an ion selective electrode) and a reference electrode. Each of the one or more working electrodes may include a membrane or surface that selectively interacts with a charged ion of interest (e.g., the analyte), causing the accumulation of a charge potential on each of the one or more working electrodes when compared to the reference electrode. The reference electrode may provide a constant half-cell potential that is unaffected by analyte concentration. A voltmeter may be used to measure the potential between the each of the one or more working electrodes and the reference electrode when no significant current flows between them. The potentiometric response may be governed by the Nernst equation such that the measured potential is proportional to a logarithm of the concentration of the analyte.

Optical biotransducers may be used in optical biosensors for signal transduction. Optical biotransducers use photons to collect information about an analyte. The detection mechanism of optical biotransducers may depend on an enzyme system that converts analyte into products that are either oxidized or reduced by a working biotransducer. For example, the evanescent field detection principle may be used in an optical biosensor system as the transduction principle. The evanescent field detection principle may allow detection of fluorophores exclusively in close proximity to a given optical biotransducer and compared to a reference biotransducer to determine an analyte level or concentration.

Electronic biotransducers may be based on field-effect transistors (FETs). A FET is a type of transistor that uses an electric field to control the conductivity of a channel (e.g., a region depleted of charge carriers) between two electrodes (e.g., the source and drain) in a semiconducting material. Control of the conductivity of the channel may be achieved by varying the electric field potential, relative to the source and drain electrode, at a third electrode, known as the gate. Depending on the configuration and doping of the semiconducting material, the presence of a sufficient positive or negative potential at the gate electrode can either attract charge carriers (e.g., electrons) or repel charge carriers in the conduction channel resulting in a change in drain current. Accordingly, as a target analyte accumulates on the gate electrode, a change in drain current can be determined and compared to a reference electrode to determine an analyte level or concentration.

Gravimetric biotransducers may use thin piezoelectric quartz crystals, either as resonating crystals (QCM), or as bulk/surface acoustic wave (SAW) devices. Thin polymer films may also be used. A bioreceptor with a known surface mass may be added to one or more surfaces of the gravimetric biotransducer to bind to the target analyte. A mass response of gravimetric biotransducers may be inversely proportional to the mass of the crystals and/or thin films. Acoustic waves can be projected to the gravimetric biosensor to produce an oscillatory response based on the mass of the gravimetric biotransducer. As an analyte binds to the gravimetric biotransducer, the mass and corresponding oscillatory response of the gravimetric biotransducer changes in proportion to the presence of the target analyte in the target environment. Additionally, the response of the working gravimetric biotransducer (e.g., binds to analyte) may be compared to a reference gravimetric biotransducer (e.g., does not bind to analyte) to determine an analyte level or concentration.

Examples of various types of biotransducers discussed herein are exemplary and do not provide an exhaustive list of biotransducers that may be used in the apparatus, systems, and methods described herein. In general, the apparatus, systems, and methods described herein include at least one working biotransducer (e.g., a biotransducer sensitive to a target analyte) and at least one reference biotransducer (e.g., a biotransducer that is not sensitive to the target analyte). Signals from working biotransducers may be compared to one or more baseline signals provided by one or more reference biotransducers to determine the portion of the signal from the working biotransducers that corresponds to the analyte level or concentration. However, reference biotransducers may be subject to noise, drift, or other error conditions that can impact the accuracy of sensor output results (e.g., analyte levels or concentration). To correct noise, drift, or other error conditions, apparatus, systems, and methods described herein may utilize one or more working as reference biotransducers.

The plurality of biotransducers 106 include at least one reference biotransducer 110, one or more working biotransducers 112, and at least one working as reference biotransducer 114. The reference biotransducer 110 may provide a baseline signal of the target environment 10. In general, the reference biotransducer 110 does not react to the analyte. In other words, the baseline signal provided by the reference biotransducer 110 is unaffected by the presence of the analyte in the target environment 10. Instead, the baseline signal may be representative of noise and other conditions of the target environment 10 that may impact signals provided by any of the plurality of biotransducers 106.

The one or more working biotransducers 112 may each provide an analyte signal based on a presence of the analyte in the target environment 10. The one or more working biotransducers 112 may be configured to interact with the analyte in such a way that the provided analyte signals vary in proportion to the concentration of the analyte in the target environment 10. For example, the one or more working biotransducers 112 may include a bioreceptor that causes molecules of the analyte to accumulate on the one or more working biotransducers 112 in proportion to the concentration of the analyte in the target environment 10. Further, for example, the one or more working biotransducers 112 may induce and/or detect optical or electromagnetic waves when in the presence of the analyte and in proportion to the concentration of the analyte. Accordingly, the analyte signals may vary in proportion to the concentration of the analyte in the target environment 10.

The at least one working as reference biotransducer 114 may provide at least one error correction signal of the target environment 10. The at least one working as reference biotransducer 114 may include a reference-like biotransducer. In other words, the at least one working as reference biotransducer 114 may be configured to be substantially identical to or have the same nominal specifications as the reference biotransducer 110. For example, the at least one reference biotransducer 110 may be a silver/silver-chloride electrode and the at least one working as reference biotransducer 114 may also be a silver/silver-chloride electrode. Error correction signals provided by reference-like biotransducers may be used to correct for noise, drift, biotransducer variation, or other error conditions that may affect reference biotransducers.

Alternatively, or additionally, the at least one working as reference biotransducer 114 may include a blank biotransducer. A blank biotransducer may be an electrode that responds to background noise of the target environment 10 but does not otherwise react (chemically or otherwise) to the target environment. For example, the at least one working as reference biotransducer 114 may be a blank electrode. Error correction signals provided by blank biotransducers may be used to correct for background noise that may affect reference biotransducers.

The at least one working as reference biotransducer 114 may include a plurality of working as reference biotransducers each configured to provide an error correction signal of the target environment 10. Additionally, the plurality of working as reference biotransducers 114 may include at least two reference-like biotransducers, at least two blank biotransducers, or at least one reference-like biotransducer and at least one blank biotransducer.

In some embodiments, the reference biotransducer 110 may optionally include or be operatively coupled to a counter electrode 111. Generally, a counter electrode may refer to an element used as a current path to complete a circuit of an analyte sensor apparatus that uses current flow between transducers or electrodes to sense an analyte. For example, the counter electrode 111 may be used for cyclic voltammetry, linear sweep voltammetry, and other electrochemical techniques. The counter electrode 111 may be operatively coupled to a power supply to provide a voltage or current within the target environment 10 or to other components of the analyte sensor apparatus 102. Accordingly, power supplied to the plurality of biotransducers 106 may be reduced. Similarly, a reduction in power requirements may allow a reduction in the size of the plurality of biotransducers 106. For example, each of the plurality of biotransducers 106 may include an electrode for sensing a voltage and/or a current. Such voltage or current may be supplied primarily by the counter electrode 111 instead of the electrodes of the at least one reference biotransducer 110, the one or more working transducers 112, or the at least one working as reference biotransducer 114. Additionally, the counter electrode 111 may act as a blank biotransducer to provide noise or drift cancellation.

The analyte sensor apparatus 102 may also include a controller 116 operatively coupled to the plurality of biotransducers 106. The controller may be configured to receive the baseline signal provided by the at least one reference biotransducer 110, the one or more analyte signals provided by the one or more working biotransducers 112, and the error correction signal provided by the at least one working as reference biotransducer 114. The controller 116 may receive signals from the plurality of biotransducers 106 via any suitable wired or wireless connection.

The controller 116 may further be configured to provide one or more analyte levels based on the baseline signal and the one or more analyte signals. The one or more analyte levels may be provided or determined based on a difference between the baseline signal and each of the one or more analyte signals.

Still further, the controller 116 may be configured to provide an error correction factor based on the baseline signal and the at least one error correction signal. The error correction factor may be provided or determined based on a difference between the baseline signal and the at least one error correction signal. Additionally, or alternatively, the error correction factor may be provided or determined based on any subtraction or differential method such as digital signal processing, noise subtraction, blind spot suppression, etc.

The controller 116 may further be configured to output one or more adjusted analyte levels based on the one or more analyte levels and the at least one error correction factor. Each of the one or more adjusted analyte levels may be a difference between an analyte level and the at least one error correction factor. In some embodiments, each of the one or more adjusted analyte levels are output by a difference amplifier that receives a provided analyte level and at least one error correction factor as inputs. In some embodiments, the controller 116 may determine the one or more adjusted analyte levels (e.g., using digital logic, one or more processors, etc.) based on the one or more analyte levels and the at least one error correction factor.

In some embodiments, the controller 116 may be an analog controller that includes one or more resistors, capacitors, operational amplifiers, comparators, filters, differential amplifiers, or other analog circuitry to provide various outputs without the use of digital circuitry, digital logic, or a processor. Such analog controller may be configured to provide one or more analyte levels based on the baseline signal and the one or more analyte signals. For example, a differential amplifier may provide a signal representative of the analyte level by providing the difference between an analyte signal and the baseline signal. Additional analogue circuitry may be used to provide the error correction factor and output the adjusted analyte levels.

In some embodiments, the controller 116 may additionally or alternatively include one or more processors, logic gates, or other digital circuitry to determine analyte levels, error correction factors, and adjusted analyte levels.

The controller 116 may include data storage for data storage and access to processing programs or routines that may be employed to carry out the techniques, processes, and algorithms for detecting an analyte in a target environment. For example, processing programs or routines may include programs or routines for determining one or more analyte levels, determining error correction factors, determining adjusted analyte levels, filtering background noise, computational mathematics, matrix mathematics, Fourier transforms, compression algorithms, calibration algorithms, inversion algorithms, signal processing algorithms, normalizing algorithms, deconvolution algorithms, averaging algorithms, standardization algorithms, comparison algorithms, vector mathematics, or any other processing required to implement one or more embodiments as described herein.

The analyte sensor apparatus 102 may also include a communication interface 118 to communicate with one or more external devices. The communication interface 118 may include any suitable hardware or devices to provide wired or wireless communication with the one or more external devices. For example, the communication interface 118 may include one or more of a receiver, transmitter, transceiver, ethernet port, Universal Serial Bus (USB) port, cables, controller, or other device to facilitate wired or wireless communication. The communication interface 118 may facilitate communication using any suitable protocol or protocols. For example, the communication interface 118 may utilize Ethernet, Recommended Standard 232, Universal Asynchronous Receiver Transmitter or Universal Synchronous Asynchronous Receiver Transmitter (UART/USART), USB, BLUETOOTH, Wi-Fi, Near Field Communication (NCF), etc. The communication interface 118 may allow communication between the analyte sensor apparatus 102 and a computing apparatus such as the computing apparatus 104.

The system 100 may also include the computing apparatus 104. The computing apparatus 104 may be, for example, any fixed or mobile computer system (e.g., a personal computer, a tablet computer, a mobile device, a cellular phone, a wearable device, etc.). The exact configuration of the computing apparatus 104 is not limiting and essentially any device capable of providing suitable computing capabilities and control capabilities (e.g., control analyte sensing of the analyte sensor apparatus 102, the acquisition of data such as sensor data, determination of various parameters such as analyte levels, error correction factors, adjusted analyte levels, etc.) may be used. Further, various peripheral devices, such as a computer display, mouse, keyboard, memory, printer, scanner, etc. are contemplated to be used in combination with the computing apparatus 104.

The computing apparatus 104 may be operatively coupled to the analyte sensor apparatus 102. For example, the computing apparatus 104 may be operatively coupled to the analyte sensor apparatus 102 via analog electrical connections, digital electrical connections, wireless connections, bus-based connections, network-based connections, internet-based connections, etc. The computing apparatus 104 can transmit data to and receive data from the analyte sensor apparatus 102. Data received from the analyte sensor apparatus 102 may include, for example, analyte signals, baseline signals, error correction signals, analyte levels, error correction factors, adjusted analyte levels, biotransducer status information, sensor parameters, etc. The computing apparatus 104 may be configured execute processes and methods described herein using data received from the analyte sensor apparatus 102. For example, the computing apparatus may be configured to determine analyte levels, correction factors, adjusted analyte levels, etc. Data transmitted by the computing apparatus 104 to the analyte sensor apparatus 102 may include commands, sensor settings, thresholds, parameters, etc.

Additionally, the analyte sensor apparatus 102 and the computing apparatus 104 may each include display apparatus 120, 124, respectively, that may be configured to display data. For example, the display apparatus 120, 124 may be configured to display one or more of the analyte levels, error correction factors, adjusted analyte levels, biotransducer status information, sensor parameters, etc. The display apparatus 120, 124 may include any apparatus capable of displaying information to a user, such as a graphical user interface 122, 126 including one or more metrics indicative of cardiac conduction system therapy benefit, one or more metrics indicative of analyte signals, baseline signals, error correction signals, analyte levels, error correction factors, adjusted analyte levels, biotransducer status information, sensor parameters, textual instructions, graphical depictions the target environment, graphical depictions or actual images of the plurality of biotransducers 106, etc. Further, the display apparatus 120, 124 may include a liquid crystal display, an organic light-emitting diode screen, a touchscreen, a cathode ray tube display, etc.

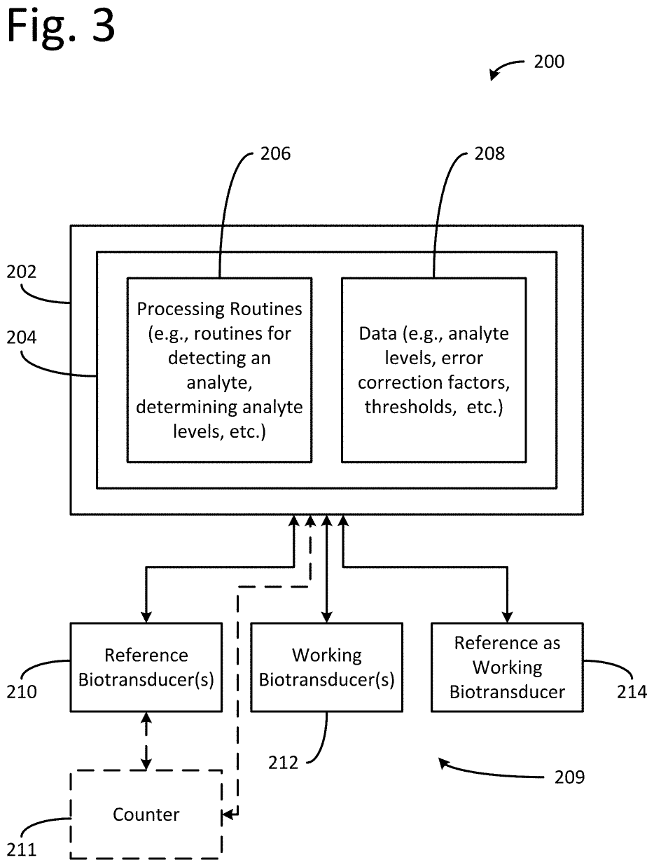

A schematic block diagram of another analyte sensing system 200 according to embodiments described herein is shown in FIG. 3. The analyte sensing system 200 may include a computing apparatus or processor 202 and a plurality of biotransducers 209. Generally, the plurality of biotransducers 209 may be operatively coupled to the computing apparatus 202 and may include any suitable circuits or devices configured to provide signals for detecting an analyte in a target environment (e.g., baseline signals, analyte signals, and error condition signals) similar to the plurality of biotransducers 106 of FIGS. 1 and 2. For example, the plurality of biotransducers 209 may include one or more electrochemical biotransducers, optical biotransducers, electronic biotransducers, gravimetric/piezoelectric biotransducers, pyroelectric biotransducers, blank biotransducers, reference-like biotransducers, bioreceptors (e.g., nucleic acids, cells, antibodies, enzymes, etc.), electrical interfaces (e.g., field effect transistors, nanowire arrays, nanoparticles, electrodes, etc.), optical interfaces, etc.

The plurality of biotransducers 209 includes at least one reference biotransducer 210, at least one working biotransducer 212, and at least one reference as working biotransducer 214. The at least one reference biotransducer 210 is configured to provide a baseline signal of the target environment and may include any of the features described herein with regard to reference biotransducer 110. The at least one working biotransducer 212 is configured to provide an analyte signal based on the presence of the analyte in the target environment and may include any of the features described herein with regard to working biotransducer 112. The at least one reference as working biotransducer 214 is configured to provide at least one error correction signal of the target environment and may include any of the features described herein with regard to working as reference biotransducer 210.

The analyte sensing system 200 may optionally include a counter electrode 211. The at least one reference biotransducer 210 may include or be operatively coupled to the counter electrode 211. The counter electrode 211 may be configured to provide power to the plurality of biotransducers 209 and may include any of the features described herein with regard to counter electrode 111. Additionally, the counter electrode 211 may be configured to act as a blank biotransducer for noise or drift cancelation.

Further, the computing apparatus 202 includes data storage 204. Data storage 204 allows for access to processing programs or routines 206 and one or more other types of data 208 that may be employed to carry out the techniques, processes, and algorithms for charging a battery or one or more electrochemical cells. For example, processing programs or routines 206 may include programs or routines for providing/determining analyte levels, providing/determining error correction factors, outputting/determining adjusted analyte levels, filtering background noise, transmitting data, receiving data, determining thresholds, computational mathematics, matrix mathematics, Fourier transforms, compression algorithms, calibration algorithms, image construction algorithms, inversion algorithms, signal processing algorithms, normalizing algorithms, deconvolution algorithms, averaging algorithms, standardization algorithms, comparison algorithms, vector mathematics, or any other processing required to implement one or more embodiments as described herein.

Data 208 may include, for example, signal data, analyte level data, baseline data, error correction factor data, biotransducer settings/parameters, calibration data, resistance calculations, device settings, error bit states, historical data, thresholds, arrays, meshes, grids, variables, counters, statistical estimations of accuracy of results, results from one or more processing programs or routines employed according to the disclosure herein (e.g., detecting an analyte in a target environment, determining adjusted analyte levels, etc.), or any other data that may be necessary for carrying out the one or more processes or techniques described herein.

In one or more embodiments, the analyte sensing system 200 may be controlled using one or more computer programs executed on programmable computers, such as computers that include, for example, processing capabilities (e.g., microcontrollers, programmable logic devices, etc.), data storage (e.g., volatile or non-volatile memory and/or storage elements), input devices, and output devices. Program code and/or logic described herein may be applied to input data to perform functionality described herein and generate desired output information. The output information may be applied as input to one or more other devices and/or processes as described herein or as would be applied in a known fashion.

The programs used to implement the processes described herein may be provided using any programmable language, e.g., a high-level procedural and/or object orientated programming language that is suitable for communicating with a computer system. Any such programs may, for example, be stored on any suitable device, e.g., a storage media, readable by a general or special purpose program, computer or a processor apparatus for configuring and operating the computer when the suitable device is read for performing the procedures described herein. In other words, at least in one embodiment, the analyte sensing system 200 may be controlled using a computer readable storage medium, configured with a computer program, where the storage medium so configured causes the computer to operate in a specific and predefined manner to perform functions described herein.

The computing apparatus 202 may be, for example, any fixed or mobile computer system (e.g., a personal computer or minicomputer). The exact configuration of the computing apparatus is not limiting and essentially any device capable of providing suitable computing capabilities and control capabilities (e.g., control biotransducer output such as voltage, current, photon, or other sensing outputs; the acquisition of data, such as sensor data; etc.) may be used. Additionally, the computing apparatus 202 may be incorporated in a housing of the analyte sensing system 200. Further, various peripheral devices, such as a computer display, mouse, keyboard, memory, printer, scanner, etc. are contemplated to be used in combination with the computing apparatus 202. Further, in one or more embodiments, the data 208 (e.g., signal data, analyte level data, baseline data, error correction factor data, biotransducer settings/parameters, calibration data, etc.) may be analyzed by a user, used by another machine that provides output based thereon, etc. As described herein, a digital file may be any medium (e.g., volatile or non-volatile memory, a CD-ROM, a punch card, magnetic recordable tape, etc.) containing digital bits (e.g., encoded in binary, trinary, etc.) that may be readable and/or writeable by computing apparatus 202 described herein. Also, as described herein, a file in user-readable format may be any representation of data (e.g., ASCII text, binary numbers, hexadecimal numbers, decimal numbers, audio, graphical) presentable on any medium (e.g., paper, a display, sound waves, etc.) readable and/or understandable by a user.

In view of the above, it will be readily apparent that the functionality as described in one or more embodiments according to the present disclosure may be implemented in any manner as would be known to one skilled in the art. As such, the computer language, the computer system, or any other software/hardware that is to be used to implement the processes described herein shall not be limiting on the scope of the systems, processes or programs (e.g., the functionality provided by such systems, processes or programs) described herein.

The techniques described in this disclosure, including those attributed to the systems, or various constituent components, may be implemented, at least in part, in hardware, software, firmware, or any combination thereof. For example, various aspects of the techniques may be implemented by the computing apparatus 202, which may use one or more processors such as, e.g., one or more microprocessors, DSPs, ASICs, FPGAs, CPLDs, microcontrollers, or any other equivalent integrated or discrete logic circuitry, as well as any combinations of such components, image processing devices, or other devices. The term “processing apparatus,” “processor,” or “processing circuitry” may generally refer to any of the foregoing logic circuitry, alone or in combination with other logic circuitry, or any other equivalent circuitry. Additionally, the use of the word “processor” may not be limited to the use of a single processor but is intended to connote that at least one processor may be used to perform the techniques and processes described herein.

Such hardware, software, and/or firmware may be implemented within the same device or within separate devices to support the various operations and functions described in this disclosure. In addition, any of the described components may be implemented together or separately as discrete but interoperable logic devices. Depiction of different features, e.g., using block diagrams, etc., is intended to highlight different functional aspects and does not necessarily imply that such features must be realized by separate hardware or software components. Rather, functionality may be performed by separate hardware or software components or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed by the computing apparatus 202 to support one or more aspects of the functionality described in this disclosure.

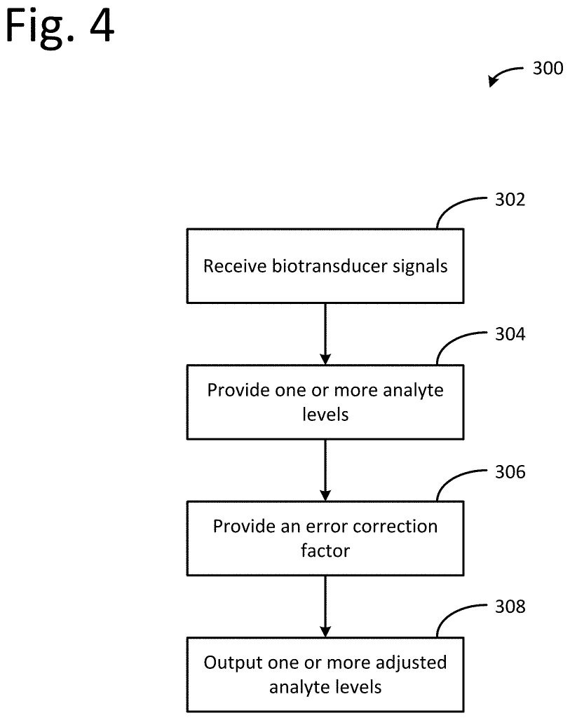

A method or process 300 of detecting an analyte in a target environment is shown in FIG. 4. Although described in regard to analyte sensor apparatus 102 of FIGS. 1 and 2, the method 300 may be carried out using any suitable analyte sensing apparatus or system.

The method 300 may include receiving the baseline signal, the one or more analyte signals, and the error correction signal 302. The baseline signal may be received from one or more reference biotransducers 110. The baseline signal may include a single baseline signal from a single reference biotransducer or a plurality of baseline signals each provided by a different reference biotransducer of the sensor apparatus 102. The baseline signal may include an average of the plurality of baseline signals. Alternatively, the controller 116 may be configured to average the plurality of baseline signals.

The one or more analyte signals may be received from one or more working biotransducers 112. The one or more analyte signals may include a single analyte signal from a single reference biotransducer or a plurality of analyte signals each provided by a different working biotransducer of the sensor apparatus 102.

The error correction signal may be received from one or more working as reference biotransducers 114. The error correction signal may include a single error correction signal from a single working as reference biotransducer or a plurality of error correction signals each provided by a different working as reference biotransducer of the sensor apparatus 102. Receiving the at least one error correction signal may include receiving a first error correction signal provided by a reference-like biotransducer and receiving a second error correction signal provided by a blank biotransducer. In some embodiments, the error correction signal may include an average of the plurality of error correction signals. Alternatively, the controller 116 may average the plurality of error correction signals.

The method 300 may further include providing one or more analyte levels based on the baseline signal and the one or more analyte signals 304. Providing the one or more analyte levels may include using the controller 116 to provide a difference between the each of the one or more analyte levels and the baseline signal.

The method 300 may further include providing an error correction factor based on the baseline signal and the at least one error correction signal 306. The error correction factor may be provided by the controller 116. The error correction factor may be a difference between the baseline signal and the at least one error correction signal. Providing the error correction factor may include filtering background noise of the baseline signal based on an error correction signal provided by a blank biotransducer. Filtering background noise of the baseline signal may provide a filtered baseline signal. The error correction factor may be provided based on the filtered baseline signal and an error correction signal provided by a reference-like biotransducer.

The method 300 may further include outputting one or more adjusted analyte levels based on the one or more analyte levels and the at least one error correction factor 308. Outputting the one or more adjusted analyte levels may include outputting a difference between the one or more analyte levels and the at least one error correction factor. The difference between the one or more analyte levels and the at least one error correction factor may be scaled or directly proportional to the electrical, magnetic, light induced artifacts, movement induced artifacts, non-faradaic currents, faradaic non-analyte induced currents, background environment conductivity, or electrochemical drift. In some embodiments, outputting the one or more adjusted analyte levels may include outputting the result of any suitable subtraction or differentiation method applied to the one or more analyte levels and the at least one error correction factor such as, for example, digital signal processing, noise subtraction, blind spot suppression, etc. Outputting the one or more adjusted analyte levels may include displaying the one or more adjusted analyte levels on the display apparatus 120 of the analyte sensor apparatus 102.

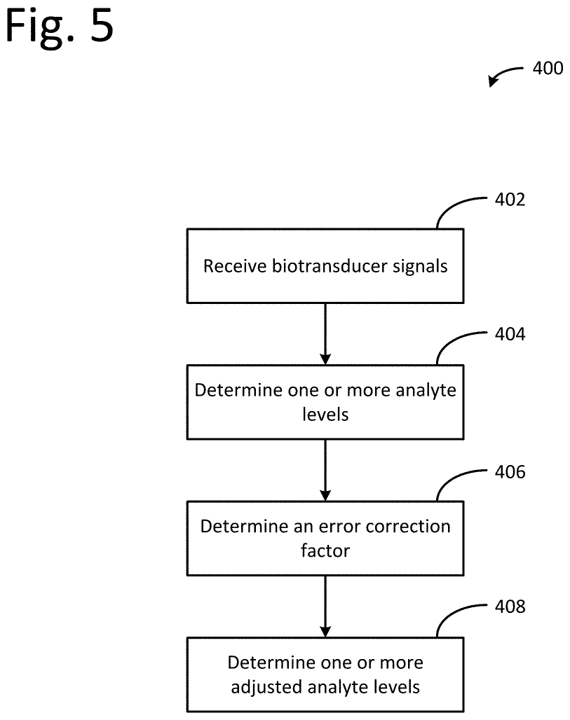

A method or process 400 of detecting an analyte in a target environment is shown in FIG. 5. Although described in regard to analyte sensor system 100 of FIGS. 1 and 2, the method 400 may be carried out using any suitable analyte sensing apparatus or system.

The method 400 may include receiving the baseline signal, the one or more analyte signals, and the error correction signal 402. The baseline signal may be received from one or more reference biotransducers 110. The baseline signal may include a single baseline signal from a single reference biotransducer or a plurality of baseline signals each provided by a different reference biotransducer of the sensor apparatus 102. The baseline signal may include an average of the plurality of baseline signals. Alternatively, the controller 116 may be configured to average the plurality of baseline signals.

The one or more analyte signals may be received from one or more working biotransducers 112. The one or more analyte signals may include a single analyte signal from a single reference biotransducer or a plurality of analyte signals each provided by a different working biotransducer of the sensor apparatus 102.

The error correction signal may be received from one or more working as reference biotransducers 114. The error correction signal may include a single error correction signal from a single working as reference biotransducer or a plurality of error correction signals each provided by a different working as reference biotransducer of the sensor apparatus 102. Receiving the at least one error correction signal may include receiving a first error correction signal provided by a reference-like biotransducer and receiving a second error correction signal provided by a blank biotransducer. In some embodiments, the error correction signal may include an average of the plurality of error correction signals. Alternatively, the controller 116 may average the plurality of error correction signals.

The method 400 may further include determining one or more analyte levels based on the baseline signal and the one or more analyte signals 404. Determining the one or more analyte levels may include using the controller 116 to provide a difference between the each of the one or more analyte levels and the baseline signal.

The method 400 may further include determining an error correction factor based on the baseline signal and the at least one error correction signal 406. The error correction factor may be determined by the controller 116 or the computing apparatus 104. The error correction factor may be determined based on a difference between the baseline signal and the at least one error correction signal. Additionally, or alternatively, determining the error correction factor may include filtering background noise of the baseline signal based on an error correction signal provided by a blank biotransducer. Filtering background noise of the baseline signal may provide a filtered baseline signal. The error correction factor may be determined based on the filtered baseline signal and an error correction signal provided by a reference-like biotransducer.

The method 400 may further include determining one or more adjusted analyte levels based on the one or more analyte levels and the at least one error correction factor 408. Determining the one or more adjusted analyte levels may include determining a difference between the one or more analyte levels and the at least one error correction factor. The difference between the one or more analyte levels and the at least one error correction factor may be scaled or directly proportional to the electrical, magnetic, light induced artifacts, movement induced artifacts, non-faradaic currents, faradaic non-analyte induced currents, background environment conductivity, or electrochemical drift. In some embodiments, determining the one or more adjusted analyte levels may include applying any suitable subtraction or differential method to the one or more analyte levels and the at least one error correction factor such as, for example, digital signal processing, noise subtraction, blind spot suppression, etc.

Additionally, the method 400 may further include displaying the one or more adjusted analyte levels. Displaying the one or more adjusted analyte levels may include displaying the one or more adjusted analyte levels on at least one of the display apparatus 120, 124 and/or graphical user interfaces 122, 126. The one or more adjusted analyte levels may be displayed as a number, graph, or other visual representation of the adjusted analyte levels.

The invention is defined in the claims. However, below there is provided a non-exhaustive list of non-limiting examples. Any one or more of the features of these examples may be combined with any one or more features of another example, embodiment, or aspect described herein.

Example Ex1: An analyte sensor apparatus for detecting an analyte in a target environment comprising: a plurality of biotransducers configured to provide a baseline signal, one or more analyte signals, and at least one error condition signal, the plurality of biotransducers comprising: at least one reference biotransducer to provide a baseline signal of the target environment; one or more working biotransducers to provide an analyte signal based on a presence of the analyte in the target environment; and at least one working as reference biotransducer to provide at least one error correction signal of the target environment; and a controller operatively coupled to the plurality of biotransducers and configured to: receive the baseline signal, the one or more analyte signals, and the error correction signal; provide one or more analyte levels based on the baseline signal and the one or more analyte signals; provide an error correction factor based on the baseline signal and the at least one error correction signal; and output one or more adjusted analyte levels based on the one or more analyte levels and the at least one error correction factor.

Example Ex2: The apparatus of example Ex1, wherein the at least one working as reference biotransducer comprises a plurality of working as reference biotransducers each configured to provide an error correction signal of the target environment.

Example Ex3: The apparatus of example Ex1, wherein the at least one working as reference biotransducer comprises a blank biotransducer and the at least one error correction signal is based on at least background noise sensed by the blank biotransducer.

Example Ex4: The apparatus of example Ex1, wherein the at least one working as reference biotransducer comprises a reference-like biotransducer and the at least one error correction signal is based on a signal provided by the reference-like biotransducer.

Example Ex5: The apparatus of example Ex1, wherein the at least one working as reference biotransducer comprises: a reference-like biotransducer configured to provide a first error signal based on the target environment; and a blank biotransducer to provide a second error signal based on background noise of the target environment; and wherein the controller is configured to provide the error correction factor based on the baseline signal, the first error signal, and the second error signal.

Example Ex6: The apparatus of example Ex1, wherein the analyte sensor apparatus comprises a communication interface configured to transmit the one or more adjusted analyte levels to an external device.

Example Ex7: The apparatus of example Ex1, wherein the analyte sensor apparatus comprises a potentiometric sensor and each of the plurality of biotransducers is an electrode.

Example Ex8: The apparatus of example Ex1, wherein the analyte sensor apparatus comprises an implantable medical device.

Example Ex9: An analyte sensing system comprising: an analyte sensor apparatus for detecting an analyte in a target environment comprising:
a plurality of biotransducers configured to provide a baseline signal, one or more analyte signals, and at least one error condition signal, the plurality of biotransducers comprising: at least one reference biotransducer to provide a baseline signal of the target environment; one or more working biotransducers to provide an analyte signal based on a presence of the analyte in the target environment; and at least one working as reference biotransducer to provide at least one error correction signal of the target environment; and a computing apparatus comprising one or more processors and operatively coupled to the analyte sensor apparatus, the computing apparatus configured to: receive the baseline signal, the one or more analyte signals, and the at least one error correction signal; determine one or more analyte levels based on the baseline signal and the one or more analyte signals; determine an error correction factor based on the baseline signal and the at least one error correction signal; and determine one or more adjusted analyte levels based on the one or more analyte levels and the error correction factor.

Example Ex10: The system of example Ex9, wherein the at least one working as reference biotransducer comprises a plurality of reference as working biotransducers each configured to provide an error condition signal of a plurality of error condition signals and wherein the computing apparatus is further configured to determine the error correction factor based on the baseline signal and an average of the plurality of error condition signals.

Example Ex11: The system of example Ex9, wherein the at least one working as reference biotransducer comprises a blank biotransducer and, to determine the error correction factor, the computing apparatus is configured to determine a background noise of the target environment based on the baseline signal and the at least one error correction signal.

Example Ex12: The system of example Ex9, wherein the at least one working as reference biotransducer comprises a reference-like biotransducer and, to determine the error correction factor, the computing apparatus is configured to determine a drift of the reference biotransducer based on the baseline signal and the at least one error correction signal.

Example Ex13: The system of example Ex9, wherein the at least one working as reference biotransducer comprises: a reference-like biotransducer configured to provide a first correction error signal based on the target environment; and a blank biotransducer to provide a second error correction signal based on background noise of the target environment; and wherein the computing apparatus is configured to: filter background noise of the baseline signal based on the second error correction signal to provide a filtered baseline signal; and determine the error correction factor based on the filtered baseline signal and the first error correction signal.

Example Ex14: The system of example Ex9, wherein the analyte sensor apparatus comprises a wearable device.

Example Ex15: The system of example Ex9, wherein the analyte sensor apparatus further comprises a communication interface configured to wirelessly transmit the baseline signal, the one or more analyte signals, and the at least one error correction signal to the computing apparatus.

Example Ex16: The system of example Ex9, wherein the computing apparatus further comprises a display and wherein the computing apparatus is further configured to display the one or more adjusted analyte levels.

Example Ex17: A method for detecting an analyte in a target environment comprising: receiving a baseline signal of the target environment, one or more analyte signals of the target environment, and at least one error correction signals from the target environment; determining one or more analyte levels based on the baseline signal and the one or more analyte signals; determining an error correction factor based on the baseline signal and the at least one error correction signals; and determining one or more adjusted analyte levels based on the one or more analyte levels and the error correction factor.

Example Ex18: The method of example Ex17, wherein determining the error correction factor comprises averaging a plurality of error correction signals provided by a plurality of working as reference biotransducers.

Example Ex19: The method of example Ex17, wherein determining the error correction factor comprises filtering background noise from the baseline signal based on an error correction signal provided by a blank biotransducer.

Example Ex20: The method of example Ex17, wherein receiving the at least one error correction signal comprises: receiving a first error correction signal provided by a reference-like biotransducer; and receiving a second error correction signal provided by a blank biotransducer; and wherein determining the error correction factor comprises: filtering background noise of the baseline signal based on the second error correction signal to provide a filtered baseline signal; and determining the error correction factor based on the filtered baseline signal and the first error correction signal. All scientific and technical terms used herein have meanings commonly used in the art unless otherwise specified. The definitions provided herein are to facilitate understanding of certain terms used frequently herein and are not meant to limit the scope of the present disclosure.

As used herein, singular forms “a,” “an” and “the” include plural referents unless the context clearly dictates otherwise. As used in this specification and the appended claims, the term “or” is generally employed in its sense including “and/or” unless the content clearly dictates otherwise. The term “and/or” means one or all of the listed elements or a combination of any two or more of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the inventive technology.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

It will be apparent to those skilled in the art that various modifications and variations can be made to the present inventive technology without departing from the spirit and scope of the disclosure. Since modifications, combinations, sub-combinations and variations of the disclosed embodiments incorporating the spirit and substance of the inventive technology may occur to persons skilled in the art, the inventive technology should be construed to include everything within the scope of the appended claims and their equivalents.

What is claimed is:

1. An analyte sensor apparatus for detecting an analyte in a target environment comprising:
a plurality of biotransducers configured to provide a baseline signal, one or more analyte signals, and at least one error condition signal, the plurality of biotransducers comprising:
at least one reference biotransducer to provide a baseline signal of the target environment;
one or more working biotransducers to provide an analyte signal based on a presence of the analyte in the target environment; and
at least one working as reference biotransducer to provide at least one error correction signal of the target environment; and
a controller operatively coupled to the plurality of biotransducers and configured to:
receive the baseline signal, the one or more analyte signals, and the error correction signal;
provide one or more analyte levels based on the baseline signal and the one or more analyte signals;
provide an error correction factor based on the baseline signal and the at least one error correction signal; and
output one or more adjusted analyte levels based on the one or more analyte levels and the at least one error correction factor.

2. The apparatus of claim 1, wherein the at least one working as reference biotransducer comprises a plurality of working as reference biotransducers each configured to provide an error correction signal of the target environment.

3. The apparatus of claim 1, wherein the at least one working as reference biotransducer comprises a blank biotransducer and the at least one error correction signal is based on at least background noise sensed by the blank biotransducer.

4. The apparatus of claim 1, wherein the at least one working as reference biotransducer comprises a reference-like biotransducer and the at least one error correction signal is based on a signal provided by the reference-like biotransducer.

5. The apparatus of claim 1, wherein the at least one working as reference biotransducer comprises:
a reference-like biotransducer configured to provide a first error signal based on the target environment; and
a blank biotransducer to provide a second error signal based on background noise of the target environment; and
wherein the controller is configured to provide the error correction factor based on the baseline signal, the first error signal, and the second error signal.

6. The apparatus of claim 1, wherein the analyte sensor apparatus comprises a communication interface configured to transmit the one or more adjusted analyte levels to an external device.

7. The apparatus of claim 1, wherein the analyte sensor apparatus comprises a potentiometric sensor and each of the plurality of biotransducers is an electrode.

8. The apparatus of claim 1, wherein the analyte sensor apparatus comprises an implantable medical device.

9. An analyte sensing system comprising:
an analyte sensor apparatus for detecting an analyte in a target environment comprising:
a plurality of biotransducers configured to provide a baseline signal, one or more analyte signals, and at least one error condition signal, the plurality of biotransducers comprising:
at least one reference biotransducer to provide a baseline signal of the target environment;
one or more working biotransducers to provide an analyte signal based on a presence of the analyte in the target environment; and
at least one working as reference biotransducer to provide at least one error correction signal of the target environment; and
a computing apparatus comprising one or more processors and operatively coupled to the analyte sensor apparatus, the computing apparatus configured to:
receive the baseline signal, the one or more analyte signals, and the at least one error correction signal;
determine one or more analyte levels based on the baseline signal and the one or more analyte signals;
determine an error correction factor based on the baseline signal and the at least one error correction signal; and
determine one or more adjusted analyte levels based on the one or more analyte levels and the error correction factor.

10. The system of claim 9, wherein the at least one working as reference biotransducer comprises a plurality of reference as working biotransducers each configured to provide an error condition signal of a plurality of error condition signals and wherein the computing apparatus is further configured to determine the error correction factor based on the baseline signal and an average of the plurality of error condition signals.

11. The system of claim 9, wherein the at least one working as reference biotransducer comprises a blank biotransducer and, to determine the error correction factor, the computing apparatus is configured to determine a background noise of the target environment based on the baseline signal and the at least one error correction signal.

12. The system of claim 9, wherein the at least one working as reference biotransducer comprises a reference-like biotransducer and, to determine the error correction factor, the computing apparatus is configured to determine a drift of the reference biotransducer based on the baseline signal and the at least one error correction signal.

13. The system of claim 9, wherein the at least one working as reference biotransducer comprises:
a reference-like biotransducer configured to provide a first correction error signal based on the target environment; and
a blank biotransducer to provide a second error correction signal based on background noise of the target environment; and
wherein the computing apparatus is configured to:
filter background noise of the baseline signal based on the second error correction signal to provide a filtered baseline signal; and
determine the error correction factor based on the filtered baseline signal and the first error correction signal.

14. The system of claim 9, wherein the analyte sensor apparatus comprises a wearable device.

15. The system of claim 9, wherein the analyte sensor apparatus further comprises a communication interface configured to wirelessly transmit the baseline signal, the one or more analyte signals, and the at least one error correction signal to the computing apparatus.

16. The system of claim 9, wherein the computing apparatus further comprises a display and wherein the computing apparatus is further configured to display the one or more adjusted analyte levels.

17. A method for detecting an analyte in a target environment comprising:
receiving a baseline signal of the target environment, one or more analyte signals of the target environment, and at least one error correction signals from the target environment;
determining one or more analyte levels based on the baseline signal and the one or more analyte signals;
determining an error correction factor based on the baseline signal and the at least one error correction signals; and
determining one or more adjusted analyte levels based on the one or more analyte levels and the error correction factor.

18. The method of claim 17, wherein determining the error correction factor comprises averaging a plurality of error correction signals provided by a plurality of working as reference biotransducers.

19. The method of claim 17, wherein determining the error correction factor comprises filtering background noise from the baseline signal based on an error correction signal provided by a blank biotransducer.

20. The method of claim 17, wherein receiving the at least one error correction signal comprises:
receiving a first error correction signal provided by a reference-like biotransducer; and
receiving a second error correction signal provided by a blank biotransducer; and
wherein determining the error correction factor comprises:
filtering background noise of the baseline signal based on the second error correction signal to provide a filtered baseline signal; and
determining the error correction factor based on the filtered baseline signal and the first error correction signal.
